(19) United States
(12) Patent Application Publication
(10) Pub. No.: US 2010/0249583 A1
(43) Pub. Date: Sep. 30, 2010

(54) Universal Intraoperative Radiation Detection Probe

(75) Inventors: Marlin O. Thurston, Columbus, OH (US); Richard B. Thurston, (US)

Correspondence Address: MUELLER AND SMITH, LPA;MUELLER-SMITH BUILDING, 7700 RIVERS EDGE DRIVE, COLUMBUS, OH, 43235, US

(73) Assignee: ACTIS, LTD, Columbus, OH (US)

(21) Appl. No.: 12/730,324

(22) Filed: Mar. 24, 2010

Related U.S. Application Data
(60) Provisional application No. 61/162,768, filed on Mar. 24, 2009.

(51) Int. Cl. A61B 6/00; G01T 1/161; G01T 1/166; G01T 1/202; G01T 1/24; G01T 7/00

(52) U.S. Cl. 600/431; 250/370.02; 250/393

(57) ABSTRACT

A radiation-detecting probe instrument has a forward working portion housing a radiation detector and a rearward user directed portion, and is in communication with a control assembly for processing and outputting signals received from the radiation detector correlative to a located radionuclide source emitting energy above about 80 KeV. The disclosed probe instrument forward portion has an annular housing having a radiation transparent tip. The radiation detector is disposed behind the radiation transparent tip. A K alpha radiation emitting wafer (e.g., Pb) wafer is disposed between the radiation transparent tip and the radiation detector. A radiation resistant (e.g., W) shield is disposed between the annular housing and the radiation detector and the Pb wafer. Radiation emitted from the radionuclide source strikes the Pb wafer causing the Pb wafer to emit K alpha radiation, which strikes the radiation detector for generating signals for communication the said control assembly.

### CROSS-REFERENCE TO RELATED APPLICATIONS

This application claims benefit of priority on provisional application Ser. No. 61/162,768, filed on Mar. 24, 2009, the disclosure of which is expressly incorporated herein by reference.

### STATEMENT REGARDING FEDERALLY SPONSORED RESEARCH

Not applicable.

### BACKGROUND

The present disclosure relates to the detection of radiation and more particularly to the in vivo detection of radiation sources bound to target tissue.

The concept of radioguided surgery was developed over 60 years ago. “Radioguided surgery” involves the use of a radiation detection probe system for the intraoperative detection of radionuclides. Today, it is a well-recognized tool for use in the surgical management of cancer. It also is used as a diagnostic tool, for example, for intraoperative lymphatic mapping, where a radiotracer is injected at the site of skin cancer and a radiation probe used to trace the movement of the radiotracer to the sentinel node for its removal. Regardless of whether surgery is involved, the use of a hand-held probe to locate radionuclides has many clinical applications, especially in the location and differentiation of neoplastic tissue.

An excellent review of radioguided surgery using gamma detection is by Povoski, et al., “A comprehensive overview of radioguided surgery using gamma detection probe technology”, World Journal of Surgical Oncology 2009, 7:11 (see also http://www.wjso.com/content/7/11), the disclosure of which is expressly incorporated herein by reference. Radionuclides and preferential locators are extensively reviewed in this article. A “preferential locator” is an agent that selectively and specifically binds to target tissue, which usually is neoplastic or cancerous. A preferential locator can be biologic (e.g., an antibody) or chemical, optionally radioactive. This article also mentions beta radionuclides and positron emitting radionuclides.

For each different source of radiation, a different probe often is designed and used. Different radiation sensitive crystals often are housed within each of these different probe constructions. Unfortunately, no probe capable of detecting virtually any radionuclide source has been developed. It is to such a universal probe that the present disclosure is addressed.

### BRIEF SUMMARY

A radiation-detecting probe instrument has a forward working portion housing a radiation detector and a rearward user directed portion. The probe instrument is in communication with a control assembly for processing and outputting signals received from the radiation detector correlative to an in vivo located radionuclide source emitting energy above about 100 KeV. The disclosed probe instrument forward portion has an annular housing having a radiation transparent tip. The radiation detector is disposed behind the radiation transparent tip. A Pb wafer is disposed between the radiation transparent tip and the radiation detector. A W shield is disposed adjacent to the radiation detector on the side opposite the Pb wafer. Radiation emitted from the in vivo radionuclide source strikes the Pb wafer causing the Pb wafer to emit Kα₁ radiation, which strikes the radiation detector for generating signals for communication the control assembly.

Another disclosed aspect is a method for detecting an external imaging radionuclide source emitting energy above about 80 KeV, wherein the external imaging radionuclide source is bound to a preferential locator that binds to neoplastic tissue. Initially, the external imaging radionuclide source bound preferential locator is administered to a patient suspected of having neoplastic tissue. The patient then is subjected to external imaging. Finally, the patient also is surgically accessed and the probe of claim 1 is used to locate said external imaging radionuclide source and, hence, neoplastic tissue in the patient.

Advantages of the disclosed probe include the ability to detect any radionuclide source have an energy emission of greater than about 80 KeV. Another advantage is the probe's ability to detect alpha emissions, gamma emissions, positron annihilation emissions, etc. A further advantage is the ability of the disclosed probe to be used to detect radiation sources in vivo where the radiation source was used previously for external imaging, such as, for example, PET or the like scanning. These and other advantages will be apparent to those skilled in the art based on the disclosure set forth herein.

### BRIEF DESCRIPTION OF THE DRAWINGS

For a fuller understanding of the nature and advantages of the present device, reference should be had to the following detailed description taken in connection with the accompanying drawings, in which:

FIG. 1 graphically displays KeV versus radiation counts recorded by a multichannel analyzer using a conventional probe (CZT) fitted with only a Cd—Zn—Te crystal and using the disclosed probe fitted with a Cd—Zn—Te crystal and lead wafer (CZT and Lead) to produce and detect Kα₁ radiation using a ^{99m}Tc radiation source;

FIG. 2 graphically displays KeV versus radiation counts recorded by a multichannel analyzer using a conventional probe (CZT) fitted with only a Cd—Zn—Te crystal and using the disclosed probe fitted with a Cd—Zn—Te crystal and lead wafer (CZT and Lead) to produce and detect Kα₁ radiation using a ³¹I radiation source;

FIG. 3 graphically displays KeV versus radiation counts recorded by a multichannel analyzer using a conventional probe (CZT) fitted with only a Cd—Zn—Te crystal and using the disclosed probe fitted with a Cd—Zn—Te crystal and lead wafer (CZT and Lead) to produce and detect Kα₁ radiation using a ¹⁸F radiation source;

FIG. 4 illustrates a representative probe tip for detecting Kα₁ radiation;

FIG. 5 illustrates a probe tip connected to a control unit by a cable;

FIG. 6 illustrates a crystal assembly for use in the disclosed probe;

FIG. 7 illustrates a probe tip embodiment for detecting Kα₁ radiation;

FIG. 8 is a block diagram of the components of Kα₁ radiation probe;

FIG. 9 is a block diagram of the components of another Kα₁ radiation probe embodiment;

FIGS. 10 and 11 represent the electrical circuits for the charge pre-amplifier for the disclosed Kα₁ radiation probe; and

FIG. 12 is the circuit diagram for the pre-amplifier for the disclosed Kα₁ radiation probe.

These drawings will be described in further detail below.

### DETAILED DESCRIPTION

### Kα1 Fluorescing Materials

L to K transitions produce “K alpha” emission. Because there are several energy sublevels in the L from which electrons can drop down to fill in the K-shell, there are in fact “K alpha 1” and “K alpha 2” peaks which are very close to one another in energy. For present purposes, either peak is acceptable. Elements, which are useful for present purposes, should be relatively inexpensive, safe to handle, and convenient to handle. Kαᵢ radiation can be generated in easy to detect amounts from, for example, Pb, Bi, TI, Hg, and the like elements. For cost, safety, and handling purposes, Pb is an element of choice for present purposes.

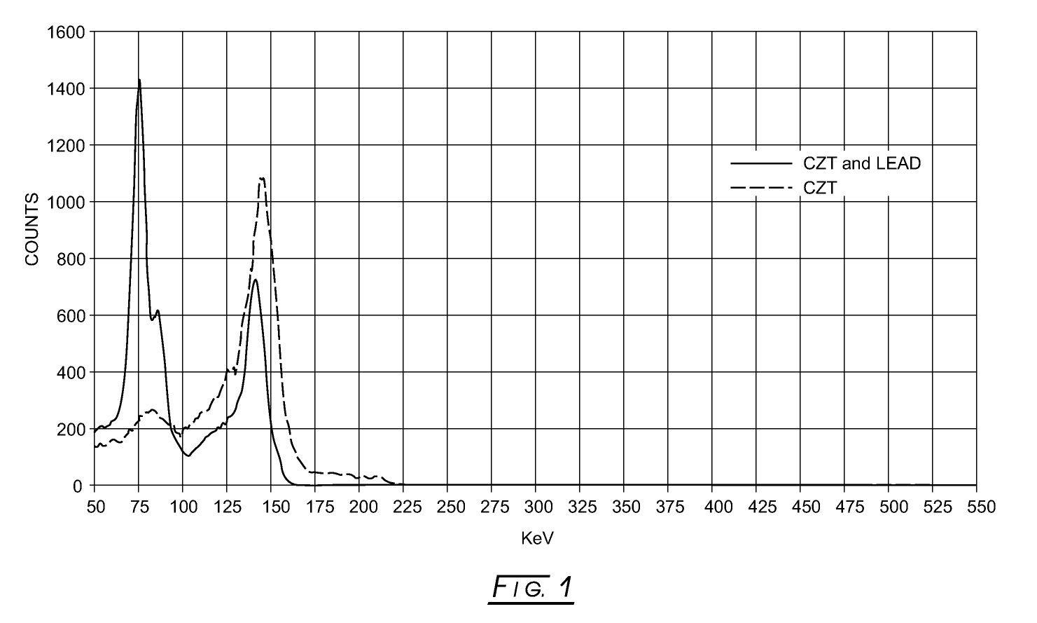

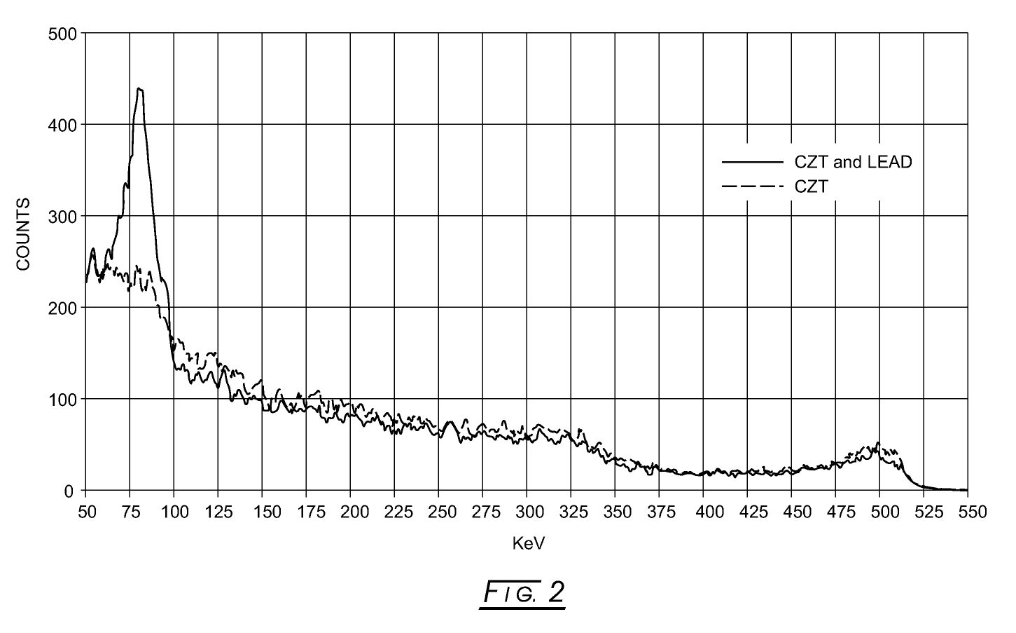

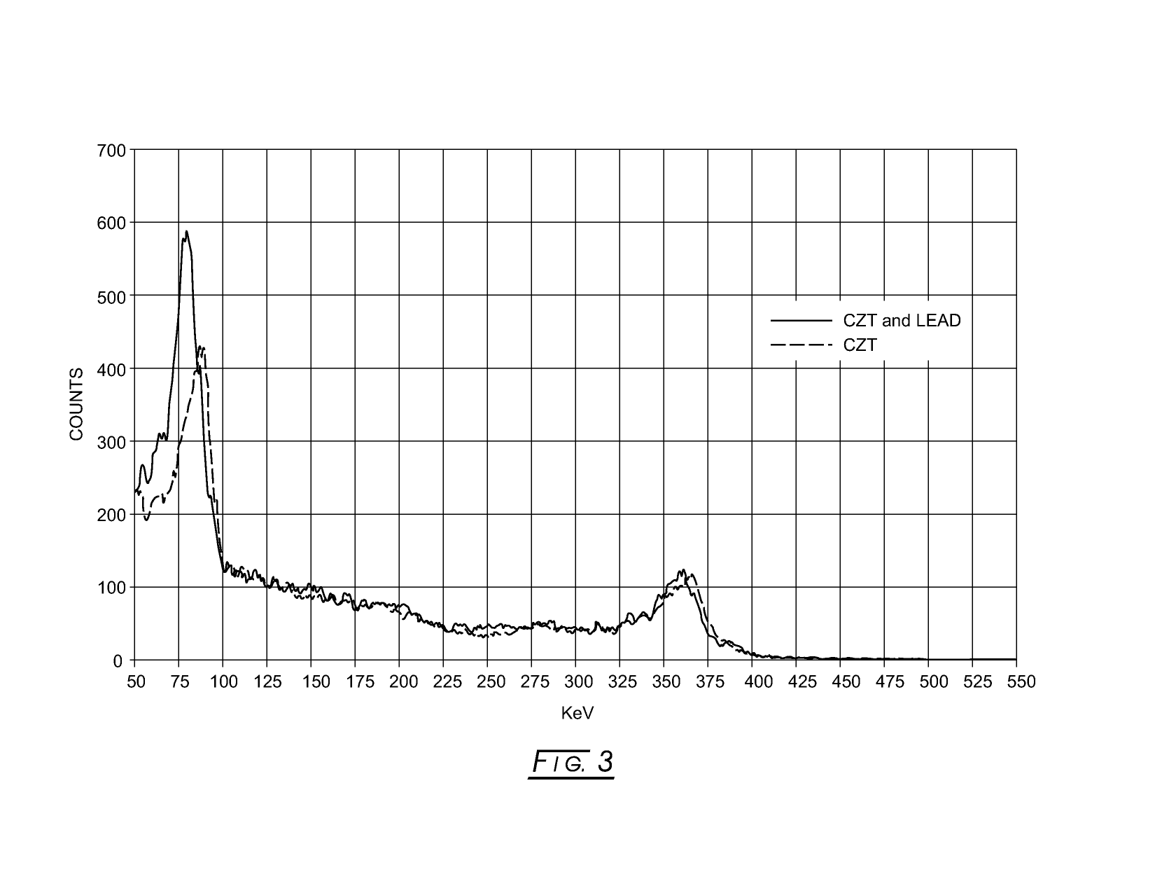

Referring to FIGS. 1-3, multichannel analyzer data collected with a convention probe (labeled, “CZT”) and with the novel Kαᵢ radiation probe (labeled, “CZT and Lead”) are displayed for ^{99m}Tc (FIG. 1), ¹³¹I (FIG. 2), and ¹⁸F (FIG. 3). ^{99m}Tc provides a peak at just below about 150 KeV using a conventional radiation detection probe, while the disclosed Kα1 radiation probe provides its characteristic peak at just above about 75 KeV. ¹³¹I provides peaks at just below about 80 KeV and at about 360 KeV using a conventional radiation detection probe, while the disclosed Kα1 radiation probe provides its characteristic peak at just above about 75 KeV. ¹⁸F provides a peak at just below about 500 KeV using a conventional radiation detection probe, while the disclosed Kα1 radiation probe provides its characteristic peak at just above about 75 KeV.

By windowing out (i.e., excluding) signals above about 80 KeV, or above about 100 KeV, direct radiation peaks of the radioisotope of interest are excluded from being detected and counted to the exclusive detection and counting of Kα1 radiation, which is the same about 75 KeV for each and every radioisotope; thus, permitting the crystal package, probe tip, circuitry, and analytical tools to be optimized to only the Kα1 radiation signal regardless of isotope. Windowing out (excluding) signals below about 50 KeV can be practiced also. A truly universal isotope detection probe, thus, is revealed.

### Radionuclides

Radionuclides useful for present purposes can generate positron emission, gamma radiation, beta radiation, or the like. Practical animal uses, however, limit the radionuclides to those that are approved for animal (including human) use. Examples of PET detectable labels include, for example, ¹⁵O, ¹³N, ¹¹C, ¹⁸F, ¹²⁴I, and ⁸²Rb. Gamma emitters (i.e., gamma radiation emitters) include, for example, ⁶⁷Ga, ¹¹¹In, ¹²³I, ¹³¹I, ^{99m}Tc, ⁵⁷Co, ²⁰¹Tl, and the like. Radionuclides approved for animal use and which can strike the Kα₁ fluorescing materials to generate detectable Kα1 radiation are useful for present purposes; however, the choice of radionuclide also may be affected by half-life, disposal issues, and like factors.

“Detectable” for present purposes means that the probe can detect, locate, and differentiate detector-generated Kα1 radiation from and over other (including background) sources of Kα1 radiation presented concomitantly to the probe.

### Kα1 Radiation Probe

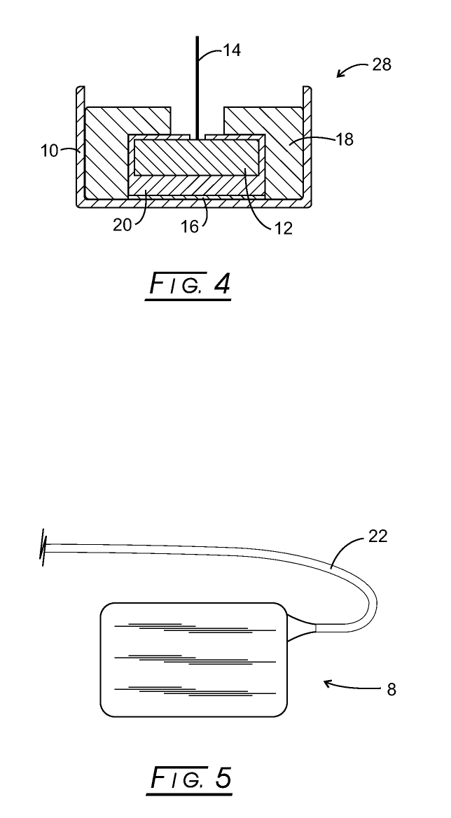

A sectional view of a representative crystal/shield/Pb sub-assembly, 8, for detecting Kα₁ radiation is illustrated in FIG. 4. An external housing, 10, can be made from Al or the like. Importantly, external housing 10 at the forward tip needs to be transparent to the radionuclide energy emitted from the radionuclide being detected. A CdTe or other suitable radiation detecting crystal, 12, is disposed within housing 10 and also serves as an anode by connection to a voltage source (e.g., 60 V) by a gold plated brass anode, 14. A cathode, 16, is disposed ahead of crystal 12 to place a, for example, 60V bias voltage across crystal 12. Surrounding crystal 12 is a radiation shield, 18, which at least shields the back surface of crystal 12 in order that detected radiation primarily pass through the forward tip for detection. This is important for spatial resolution of the probe. Suitable radiation shields should not generate significant amounts of Kα₁ radiation (compared to the amount of Kα₁ radiation entering the probe tip). Suitable materials include, then, W, Ta, Ag, Pd, Rh, Ru, Fe, Ni, Cu, Sn, Zn and the like, mixtures thereof, and alloys thereof.

Disposed between the forward tip of housing 10 and crystal 12, is a Pb wafer, 20, which generates Kα₁ radiation for detection by crystal 12. Because Pb wafer 20 is so thin, shielding by shield 18 is not necessary.

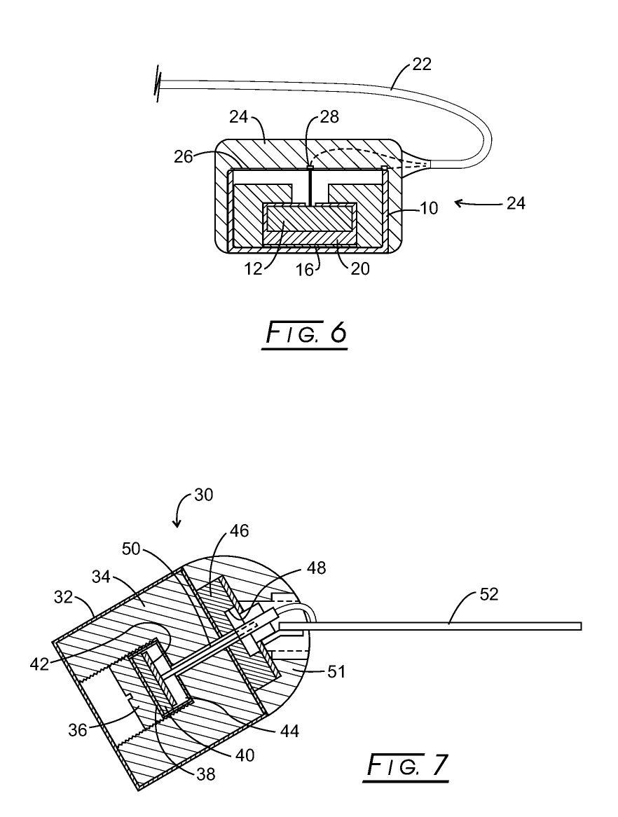

Crystal 12 disposed within probe tip 8 is connected to a control unit by a cable, 22, as illustrated in FIG. 5. In FIG. 6, crystal/shield/Pb sub-assembly (FIG. 4) is disposed within a probe tip, 24, can be made as an assembly for mounting into a tip assembly for attachment onto a hand-graspable elongate probe handle. Probe tip 24 in FIG. 6 can be manufactured at around 19 mm probe tip width by 12.5 mm height. A pre-amplifier assembly, 26, can be mounted with crystal/shield/Pb sub-assembly as illustrated in FIG. 6. An anode, 28, disposed adjacent to pre-amplifier assembly 26 completes the components within probe tip 24.

Another probe tip embodiment, 30, is illustrated in FIG. 7. An outer aluminum cap, 32, houses a tungsten shield, 34, a forward central aperture is threaded and into which is disposed the crystal sub-assembly. Such crystal sub-assembly includes (from outside to inside) a threaded retainer nut, 36, a lead plate, 38, a crystal (e.g., Cd—Zn—Te crystal), 40, W wafer, Teflon insulator, 42, and a silver K-alpha shield, 44. The back assembly includes an apertured W wafer, 46, Teflon insulator, 48, anode contact, 50, and a stainless steel housing, 51. Anode 50 is in electrical connection with a pre-amplifier assembly, 52. Inasmuch as probe tip 30 angles from the probe handle (not shown), pre-amplifier assembly 52 is angled away from probe tip 30 and follows the longitudinal axis of the probe handle.

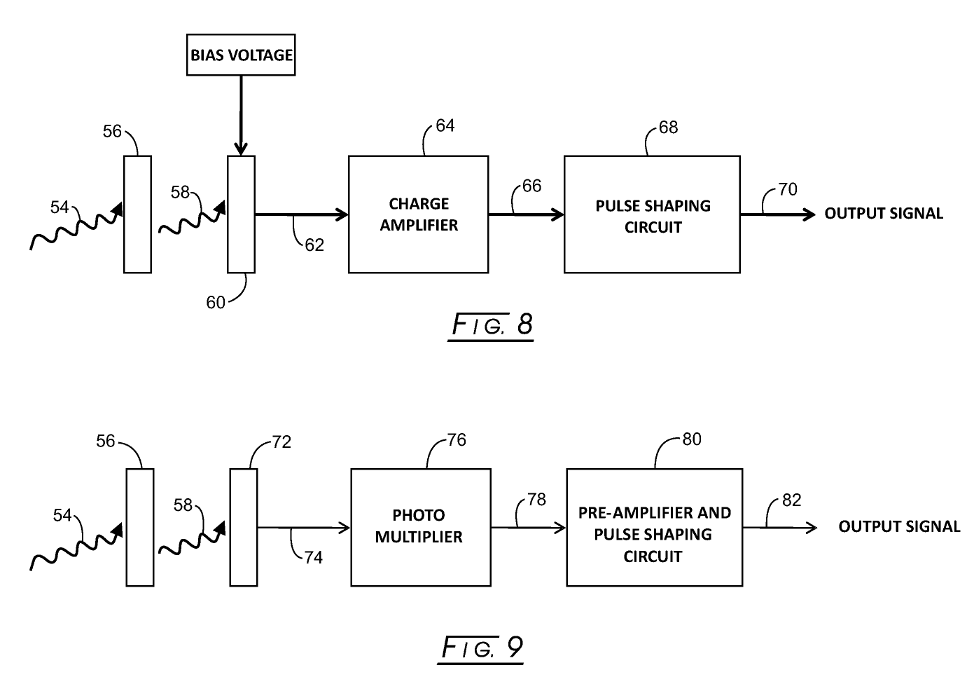

The basic components needed for the disclosed Kα₁ radiation probe and controller assemblies are illustrated in FIG. 8 for a semi-conductor crystal, such as, for example, Cd—Zn—Te crystal, and in FIG. 9 for a scintillation crystal, such as, for example, bismuth germanate. Referring initially to FIG. 8, a source of gamma radiation above about 88 KeV, 54, strikes a fluorescing plate (e.g., Pb), 56, to generate Kα₁ radiation of about 73-75 KeV (for Pb), 58, which in turn strikes a semi-conductor crystal, 60, which is held under a bias voltage. A signal, 62, generated by Cd—Zn—Te or other semi-conductor crystal 60 leads to a charge amplifier, 64, whose output signal, 66, is fed to a pulse shaping circuit, 68, which produces an output signal, 70.

In FIG. 9, gamma radiation source 54 strikes fluorescing plate 56 to produce Kα₁ radiation 58 that strikes a scintillation crystal, 72. An output, 74, from scintillation crystal 72 is fed to a photo multiplier, 76, whose output, 78, goes to a pre-amplifier and pulse shaping circuit, 80, that produces an output signal, 80.

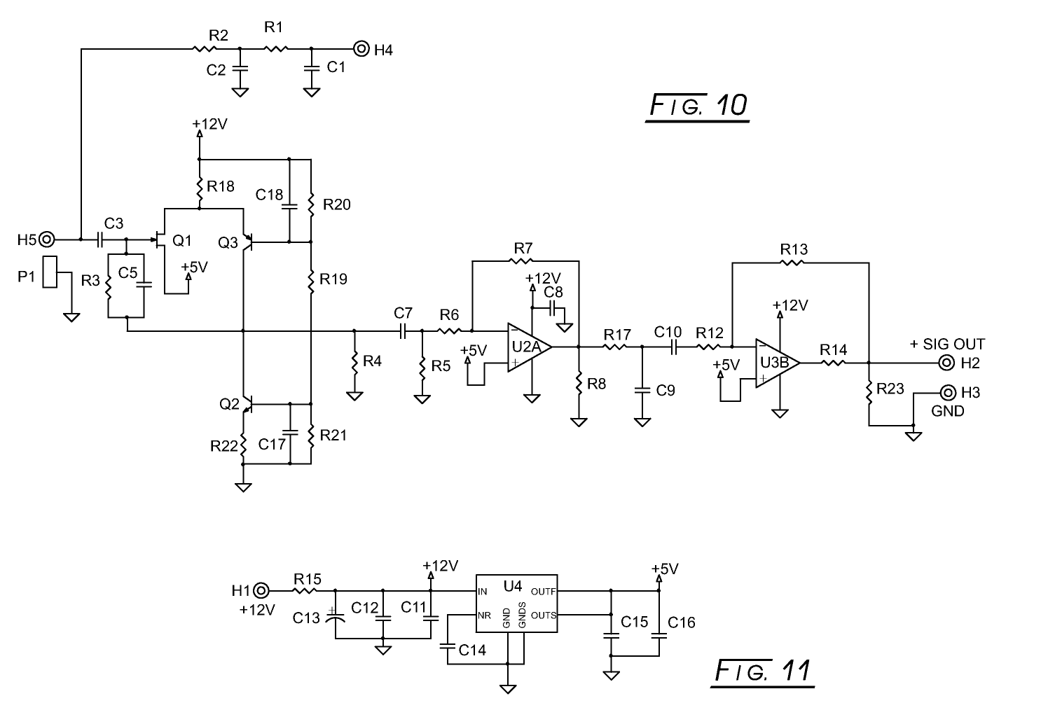

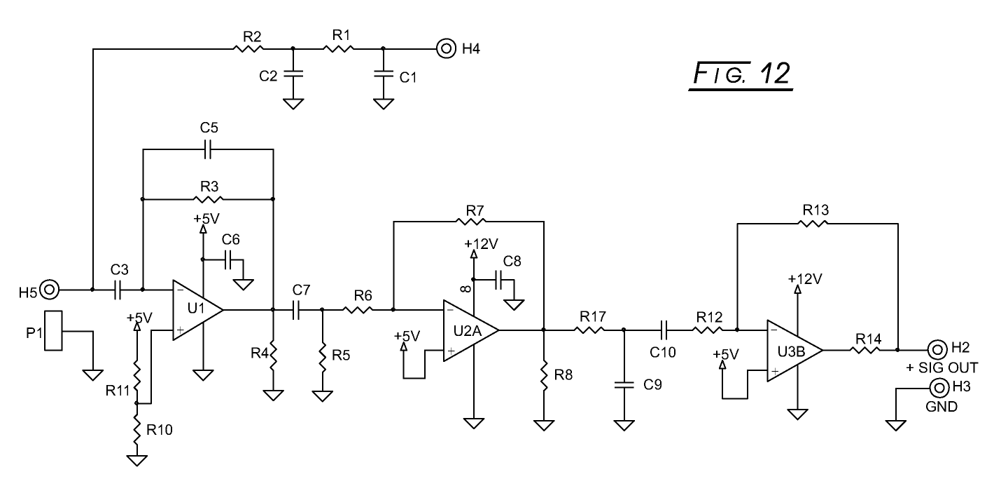

With respect to the Kα₁ radiation pre-amplifiers in FIGS. 10-12, K-alpha gamma photons are emitted from the metallic fluorescent plate when excited by a gamma energy source in excess of the electron binding energy for the inner most electron orbital. For lead, this binding energy is 88 KeV. The K-alpha emissions are given off at 73 and 75 KeV regardless of the gamma excitation energy, as long as it exceeds the electron binding energy.

The K-alpha emissions of the lower energy are trapped within the Cadmium-Zinc-Telluride crystal lattice and produce a free electron cloud by energy transfer. This free charge migrates to the high voltage anode end of the crystal. The resulting electrical signal is a voltage pulse of a few microvolts and less than a microsecond in duration. The first stage of the pre-amplifer converts this voltage pulse to a detectable level by integrating the charge of the voltage pulse. The discrete form of the circuit is described subsequently.

The high voltage DC bias is removed from the voltage pulse signal by capacitor C3. The JFET transistor, Q1, provides high input impedance and voltage to current gain by virtue of transconductance. Since the drain resistor (R18) is also connected to the emitter of the Q3 bipolar junction transistor, the change in drain current in Q1 drives a voltage change in the collectors of the Q3 and Q2 Cascode transistor pair. This three-transistor circuit provides a voltage gain of approximately 500. The R3 and C5 feedback impedance between the Q2-Q3 collectors and the gate of Q1 increases the pulse duration by integrating the charge of the voltage pulse.

The output voltage pulse (Q2-Q3 collector voltage) is further amplified in a two stage operational amplifier circuit and the rise and fall time of the pulse are set using a high pass filter (C7 and R5) and a low pass filter (R17 and C9). The total gain of the circuit is adjusted such that the final output signal is 6 millivolts per each KeV of the energy pulse interacting with the CZT crystal.

In another embodiment of the pre-amplifier circuit, the three-transistor configuration is replaced with an operational amplifier specifically designed for charge amplification. The LTC6240HV is designed with a FET input to provide the high impedance necessary to detect the CZT pulse without significant loading just as the Q1 JFET in the previous circuit. The integration is performed by the R3 and C5 feedback path, also corresponding to the previous circuit. The subsequent gain stages and filtering are identical.

In both the semi-conductor crystal embodiment and the scintillation crystal embodiment, an output signal correlative directly with the radioisotope being detected through detection of Kαᵢ radiation results. The ability to convert virtually any radioisotope signal into a constant Kαᵢ radiation signal makes the disclosed probe system unique and highly useful, especially in the detection of cell bound radioisotopes in vivo and ex vivo.

The disclosed probe can be mounted into a small assembly formed as a finger ring for use by a surgeon (“finger probe”), mounted into a thin handle assembly for laparoscopic use of the probe, or any other convenient probe construction. The following patents show various probe constructions and controller details. Many of such probe bodies and controllers find use for constructing and controlling the disclosed Kat radiation probe: U.S. Pat. Nos. 4,801,803, 4,893,013, 4,889,991, 6,070,878, 5,151,598, 5,429,133, 5,383,456, 5,441,050, 5,495,111, 5,475,219, 5,732,704, 5,857,463, 5,987,350, 5,682,888, 5,916,167, 5,928,150, 6,222,193, 6,204,505, 6,191,422, 6,218,669, 6,259,095, 6,272,373, and 6,144,876, the discloses of which are expressly incorporated herein by reference.

A prototype probe using a 19 mm CdTe crystal and Pb Kα₁ radiation generator was used to detect ¹²⁴I radiation in order to illustrate operation of the disclosed probe construction using Pb for Kα₁ radiation generation. Counts per second (cps) versus detected Kα₁ radiation (keV) is plotted in FIG. 5 for ¹²⁴I radiation. Peaks at about 511 and 603 keV can be seen. The radiation detecting window prototype probe controller was set to between 50 and 100 keV in order to detect only energy within this window. A peak of about 70 keV was detected by virtue of the Pb foil ahead of the CdTe detector crystal was seen, as illustrated in FIG. 6.

### Preferential Locators

Tumor-associated antigen (TAG-72) is a human mucin (MUC1) like glycoprotein complex with molecular weight of 10⁶ Da. It is over-expressed in several epithelial-derived cancers, including most ductal carcinomas of the breast, common epithelial ovarian carcinomas, non-small cell lung carcinomas, gastric, pancreatic, and colorectal carcinomas. Murine monoclonal antibody (B72.3) was generated using membrane-enriched extracts of human metastatic mammary carcinoma lesions, while the second generation monoclonal antibody (CC49) was generated against purified TAG-72 from colon cancer. These antibodies have been extensively evaluated in animal models and human for detection of various cancers, one of which has been approved by FDA for the detection of both colorectal and ovarian cancers with in gamma camera scanning in conjunction with computerized tomography. (¹¹¹Indium labeled B72.3 antibody, CYT-103, Cytogen).

TAG-72 antibody shows selective reactivity for human adenocarcinomas, demonstrating that 94% of colon carcinomas express the TAG-72, while normal colon epithelium does not show any reactivity to the antibody. Murine monoclonal B72.3 also reacted with cells in areas of “atypia” within adenomas. It also showed reactivity with other human carcinomas including 84% of invasive ductal breast cancer, 100% of ovarian cancers tested, and 96% lung of adenocarcinomas, while it showed only weak or no reactivity in the corresponding normal tissues except secretory endometrium.

B72.3 antibody has been evaluated in tissue culture and xenograft models. Interestingly, this antibody is not reactive to vast majority of human carcinoma cell lines in cultures due to limitations in this special configuration. However, it is highly expressed in colon cancer cell lines (e.g., LS 174T) and breast cancer cells lines (e.g., MCF-7). When these cells were grown in spheroid culture, suspension cultures or on agar, TAG-72 expression increased by 2-10 fold. Additionally, when the LS 174T cell line was injected into athymic mice to generate xenograft models, the level of TAG-72 antigen increased over 100-fold, which is similar to expression levels seen in the metastatic tumor masses from patients. I¹²⁵-labeled B72.3 was tested in xenograft mice models with LS-174 cancer cells for tumor localization.

After intravenous injection of 1.5 μCi of ¹²⁵I-labeled B72.3, 10% of injected dose per gram of body weight (% ID/g) was determined after two days. Interestingly, the total amount of ¹²⁵I-B72.3 activity in the tumor stayed constant during 30 days, while the activity in the rest of the body including blood, kidney, liver, spleen, and lung decreased significantly. For example, The % ID/g of ¹²⁵I-B72.3 in tumors stayed at 6.49% to 10.75% in 7 days period, while it decreased from 9.94% to 1.38% in blood, 1.82% to 0.34% in kidney, 2.23% to 0.37% in spleen, 5.52% to 0.75% in lung, and 1.89% to 0.37%. The distribution ratio of tumor compared to other normal organs (liver, kidney, lung) reached 18:1 at day 7, while tumor to blood ratio reached 5:1 at day 7. In xenograft models with A375 cells without TAG-72 expression, B72.3 did not show any tumor localization. In xenograft models implanting LS 174T with high levels of TAG-72, other control antibodies such as ¹²⁵I-MOPC-21 IgG did not show tumor localization either.

¹³¹I labeled B72.3 IgG has been used clinically for diagnostic imaging of colorectal, ovary, and breast cancer. The data demonstrate the specific localization of B72.3 antibody in cancer tissues in patients. After intravenous (IV) administered ¹³¹I-labeled B72.3 IgG prior to surgery, radio-localization indices (RI) were calculated by cpm of ¹³¹I-labeled antibody per gram of tumor versus cpm per gram of normal tissues. Seventy percent (99 of 142) of tumor lesions showed RI is of greater than 3 (antibody localization in tumors is 3 times greater than normal tissue). In addition, high-performance liquid chromatography (HPLC) and SDS-polyacrylamide gel electrophoresis demonstrated that the radioactivity in patient's sera was associated with intact ¹³¹I-B72.3 antibody as visualized in autoradiography or IgG peak in HPLC analysis after IV administration of dose range 0.5-20 mg. Interestingly, when ¹³¹I-labeled B72.3 IgG was administered intraperitoneally in colon cancer patients, the localization in colon tumor verse normal tissue was 70:1. However, IV administration of this labeled antibody is more efficient in targeting lymph node metastases.

¹²⁵I-labeled B72.3 also has been used for radio-immunoguided surgery (RIGS®, U.S. Pat. No. 4,782,840) with an intraoperative hand-held probe to localize the residual tumor tissue for resection. The RIGS system also has been successfully used with the B72.3 antibody for clinical colorectal cancer patients. ¹²⁵I labeled-antibody has localized 75%-80% of primary colorectal tumor lesion, and 63%-73% of metastatic lesions in lymph nodes and liver.

The second-generation antibody CC49 was generated against TAG-72 purified from colon cancer. CC49 showed higher binding affinity than B72.3 to TAG-72 in carcinomas including breast, colorectal, ovarian, and lung carcinomas, while CC49 exhibited minimum reactivity with normal tissues. When ¹²⁵I-CC49 was administered in xenograft models with colon cancer cells LS 174T, the plasma clearance was much faster than B72.3, which results in much higher tumor to normal tissue distribution ratio. For example, the tumor to blood ratio was 18.1, tumor to liver ratio 3.81, tumor to spleen ratio 16.64, tumor to kidney ratio 36.48, and tumor to lung ratio 25.82. In RIGS studies of 300 patients with colorectal cancers, CC49 was able to successfully detect tumors in 86% of patients with primary tumors and 95% of patients with recurrent tumors. In addition, clinical studies of a modified humanized antibody HuCC49ΔCH₂ with a deletion in glycosylation sites of the antibody showed similar results with CC49 in detection of colorectal cancer. See, for example, the following reported clinical trial: Pilot Study Using a Humanized CC49 Monoclonal Antibody (HuCC49ΔCH₂) to Localize Recurrent Colorectal Carcinoma Doreen M. Agnese, MD, Shahab F. Abdessalam, MD, William E. Burak, Jr., MD, Mark W. Arnold, MD, Denise Soble, RN, George H. Hinkle, RPh, Donn Young, PhD, M. B. Khazalaeli, PhD, and Edward W. Martin, Jr., MD Annals of Surgical Oncology, 11(2): 197-202; and Pharmacokinetics and Clinical Evaluation of ¹²⁵I-Radiolabeled Humanized CC49 Monoclonal Antibody (HuCC49ΔCH₂) in Recurrent and Metastatic Colorectal Cancer Patients Jim Xiao, Sara Horst, George Hinkle, Xianhua Cao, Ergun Kocak, Jing Fang, Donn Young, M. Khazaeli, Doreen Agnese, Duxin Sun, and Edward Marting, Jr., Cancer Biotherapy & Radiopharmaceuticals, Volume 20, Number 1, 2005. See also, Agnese, et al., “Pilot Study Using CC49 Monoclonal Antibody (HuCC49ΔCH₂) to Localize Recurrent Colorectal Carcinoma”, Annals of Surgical Oncology 11(2): 197-202 (“TAG-72 is an antigen expressed in several epithelial-derived cancers, including most colonic adenocarcinomas, invasive ductal carcinomas of the breast, non-small cell lung carcinomas, common epithelial ovarian carcinomas, and most pancreatic, gastric and esophageal cancers evaluated.”); Thor, et al., “Distribution of Oncofetal Antigen Tumor-associated Glycoprotein-72 Defined by Monoclonal Antibody B72.3” Cancer Research 46, 3118-3124, June 1986, (TAG-72 was shown to be expressed in several epithelial-derived cancers including 94% of colonic adenocarcinomas, 84% of invasive ductal carcinomas of the breast, 96% of non-small cell lung carcinomas, 100% of common epithelial ovarian carcinomas, as well as the majority of pancreatic, gastric and esophageal cancers evaluated. TAG-72 expression was not observed, however, in tumors of neural, hematopoietic, or sarcomatous derivation, suggesting that the TAG-72 antigen is “pancarcinoma” in nature. Appreciable monoclonal antibody B72.3 reactivity was generally not observed in adult normal tissues, with limited reactivity noted in a few benign lesions of the breast and colon. TAG-72 antigen expression was detected, however, in fetal colon, stomach, and esophagus, thus defining TAG-72 as an oncofetal antigen.”).

Both B72.3 and CC49 have demonstrated promising results in tumor detection utilizing the RIGS procedure to significantly improve patient survival rate. However, in many cases, patients have shown metastatic cancers or multiple lesions, which are not resectable. In such cases, even though the antibodies used with RIGS are able to detect the tumors, surgery cannot be employed to remove the tumors. The long half-life of ¹²⁵I, waste disposal of ¹²⁵I, and other problems associated with ¹²⁵I also make this procedure difficult for the market to accept. Other labels, such as, for example, ¹⁸F with a 110-minute half life, will not work in this procedure, because of the need to wait 21 days after antibody injection in order for non-bound antibody to clear the body.

Thus, antibody CC49, its humanized and domain deleted forms, and related TAG antibodies have been described in the literature, such as, by Xiao, et al., “Pharmocokinetics and clinical evaluation of ¹²⁵I-radiolabeled humanized CC49 monoclonal antibody (HuCC49ΔCH₂) in recurrent and metastatic colorectal cancer patients”, Cancer Biother Radiopharm, vol. 20, number 1, 2005; Fang, et al., “Population pharmocokinetics and tumor targeting of HuCC49ΔCH₂, a novel monoclonal antibody for tumor detection”, Fang, et al., J Clin Pharmacol 2007; 47:227-237; U.S. Pat. Nos. 6,418,338 and 6,760,612 (which also show peptide, lectin, and other detector molecules. See also, Slavin-Chiorini, et al., “A CDR-Grafted (Humanized) Domain-Deleted Antitumor Antibody”, Cancer Biotherapy and Radiopharmaceuticals, Volume 12, Number 5, 1997, Mary Ann Liebert, Inc. (“The MAb chosen for engineering was CC49, which is directed against a pancarcinoma antigen designated TAG-72 that is expressed on the majority of colorectal, gastric, breast, ovarian, prostate, pancreatic and lung carcinomas.”).

Yet another humanized antibody of CC49 MAb is known as V59. Gonzales, et al., “Minimizing immunogenicity of the SDR-grafted humanized antibody CC49 by genetic manipulation of the framework residues”, Molecular Immunology 40 (2003) 337-349. V59 is reported to be a fully humanized version of CC49 MAb, making it a likely choice for use in accordance with the disclosure set forth herein.

In the early 1990s investigators utilized the RIGS system to locate, differentiate and stage other types of cancer, for instance, endocrine tumors involved, inter alia, with breast, children, gastrinomas, lung and nervous system. Generally, the approach was to administer a radiolabeled somatostatin congener to assess the patient with the RIGS probe. However, before subjecting the patient to such administration, an initial determination preferably was made as to whether the radiolabeled somatostatin congener would bind to the tumor site, i.e., whether somatostatin receptors are associated with the neoplastic tissue. This was conveniently done with a wide variety of endocrine tumors, which release peptides or hormones, referred to as “biochemical markers.” In order to make this determination, initially a biochemical marker-inhibiting dose of unlabeled somatastatin congener was administered to the patient. The biochemical marker associated with the neoplastic tissue then was monitored to determine whether the administered somatostatin congener reduces the presence of the marker in the patient. If the monitored presence of the marker was reduced, then the surgeon could be confident that the neoplastic tissue or tumor contains receptors to which the somatostatin would bind. Thus, the administration of radiolabeled somatostatin congener was appropriate for such patient. If the biochemical marker associated with the neoplastic tissue was not appropriately reduced following the administration of the unlabeled somatostatin congener, then the neoplastic tissue may not be determinable by the use of radiolabeled somatostatin congener and alternative modalities of treatment would be considered, such as the use of radiolabeled antibodies. See: O'Dorisio, et al., U.S. Pat. No. 5,590,656; entitled “Application of Peptide/Cell Receptor Kinetics Utilizing Radiolabeled Somatostatin Congeners in the In Situ, In Vivo Detection and Differentiation of Neoplastic Tissue”; issued Jan. 7, 1997 and incorporated herein by reference.

In broader contexts, a locator that specifically binds a marker produced by or associated with neoplastic tissue is used in accordance with the present teachings, with antibodies and somatostatin congener being representative such locators. Broader, however, a “locator” includes a substance that preferentially concentrates at the tumor sites by binding with a marker (the cancer cell or a product of the cancer cell, for example) produced by or associated with neoplastic tissue or neoplasms. Appropriate locators today primarily include antibodies (whole and monoclonal), antibody fragments, chimeric versions of whole antibodies and antibody fragments, and humanized versions thereof. It will be appreciated, however, that single chain antibodies (SCAs, such as disclosed in U.S. Pat. No. 4,946,778, incorporated herein by reference) and like substances have been developed and may similarly prove efficacious. For example, genetic engineering has been used to generate a variety of modified antibody molecules with distinctive properties. These include various antibody fragments and various antibody formats. An antibody fragment is intended to mean any portion of a complete antibody molecule. This includes both terminal deletions and protease digestion-derived molecules, as well as immunoglobulin molecules with internal deletions, such as deletions in the IgG constant region that alter Fc mediated antibody effector functions. Thus, an IgG heavy chain with a deletion of the Fc CH2 domain is an example of an antibody fragment. It is also useful to engineer antibody molecules to provide various antibody formats. In addition to single chain antibodies, useful antibody formats include divalent antibodies, tetrabodies, triabodies, diabodies, minibodies, camelid derived antibodies, shark derived antibodies, and other antibody formats. Aptimers and peptides form yet further classes of preferential locators. All of these antibody-derived molecules are example of preferential locators.

In addition to antibodies, biochemistry and genetic engineering have been used to produce protein molecules that mimic the function of antibodies. Avimers are an example of such molecules. See, generally, Jeong, et al., “Avimers hold their own”, Nature Biotechnology Vol. 23 No. 12 (December 2005). Avimers are useful because they have low immunogenicity in vivo and can be engineered to preferentially locate to a wide range of target molecules such as cell specific cell surface molecules. Although such substances may not be subsumed within the traditional definition of “antibody”, avimer molecules that selectively concentrate at the sites of neoplastic tissue are intended to be included within the definition of preferential locator. Thus, the terms “locator” was chosen, to include present-day antibodies and equivalents thereof, such as avimers, as well as other engineered proteins and substances, either already demonstrated or yet to be discovered, which mimic the specific binding properties of antibodies in the inventive method disclosed therein.

Thus, while monoclonal antibodies can be used to advantage in the present disclosure and will be used herein to illustrate the precepts disclosed herein, as noted above, a variety of additional detector molecules for markers associated with cancer cells (TAG), are suitable for use in the present context. Thus, detector molecule should be interpreted broadly for present purposes.

While the apparatus has been described with reference to various embodiments, those skilled in the art will understand that various changes may be made and equivalents may be substituted for elements thereof without departing from the scope and essence of the disclosure. Additionally, many modifications may be made to adapt a particular situation or material to the teachings of the disclosure without departing from the essential scope thereof. Therefore, it is intended that the disclosure may not be limited to the particular embodiments disclosed, but that the disclosure will include all embodiments falling within the scope of the appended claims. In this application the US measurement system is used, unless otherwise expressly indicated. Also, all citations referred to herein are expressly incorporated herein by reference.

I claim:

1. An improved radiation-detecting probe instrument having a forward working portion housing, a radiation detector, and a rearward user directed portion, said probe instrument in communication with a control assembly for processing and outputting signals received from said radiation detector correlative to a located radionuclide source emitting energy above about 80 KeV, the improvement for detecting said radionuclide source emitting energy above about 80 KeV which comprises:
said forward portion comprising an annular housing having a radiation transparent tip, said radiation detector disposed behind said radiation transparent tip, a K alpha fluorescing radiation emitting wafer disposed between said radiation transparent tip and said radiation detector, a radiation-resistant shield disposed between said annular housing and said radiation detector and said wafer,
whereby radiation emitted from said radionuclide source strikes said K alpha radiation emitting wafer causing said wafer to emit K alpha radiation which strikes said radiation detector for generating signals for communication with said control assembly.

2. The improved radiation-detecting probe instrument of claim 1, wherein said K alpha emitting wafer is one or more of Pb, Bi, Te, or Hg.

3. The improved radiation-detecting probe instrument of claim 2, wherein said K alpha fluorescing radiation emitting wafer comprises Pb.

4. The improved radiation-detecting probe instrument of claim 1, wherein said radiation-resistant shield is one or more of W or Ag.

5. The improved radiation-detecting probe instrument of claim 4, wherein said radiation-resistant shield comprises W.

6. The improved radiation-detecting probe instrument of claim 5, wherein said K alpha fluorescing radiation emitting wafer comprises Pb.

7. The improved radiation-detecting probe instrument of claim 1, wherein said radiation detector is one or more of a semi-conductor or a scintillation crystal.

8. The improved radiation-detecting probe instrument of claim 1, wherein said semi-conductor radiation detector is a cadmium telluride crystal.

9. The improved radiation-detecting probe instrument of claim 8, wherein said semi-conductor radiation detector is a cadmium zinc telluride crystal.

10. The improved radiation-detecting probe instrument of claim 1, which is constructed as a finger probe.

11. A method for detecting said radionuclide source emitting energy above about 80 KeV, which comprises the steps of:
(a) providing a radiation-detecting probe instrument having a forward working portion housing, a radiation detector and a rearward user directed portion, said probe instrument in communication with a control assembly for processing and outputting signals received from said radiation detector correlative to a located radionuclide source emitting energy above about 80 KeV, wherein said forward portion comprises an annular housing having a radiation transparent tip, said radiation detector disposed behind said radiation transparent tip, a K alpha fluorescing radiation emitting wafer disposed between said radiation transparent tip and said radiation detector, a radiation-resistant shield disposed between said annular housing and said radiation detector and said wafer;
(b) placing said forward working portion adjacent to a suspected radionuclide source emitting energy above about 80 KeV;
(c) said radiation detector detecting K alpha radiation emitting from said K alpha fluorescing radiation emitting wafer causing said wafer and emitting electrical signals in response to detected K alpha radiation; and
(d) passing said emitted electrical signals to said control unit.

12. The method of claim 11, further comprising providing said K alpha fluorescing radiation emitting wafer to be one or more of Pb, Bi, Te, or Hg.

13. The method of claim 11, further comprising providing said radiation-resistant shield to be one or more of W or Ag.

14. The method of claim 13, further comprising providing said K alpha fluorescing radiation emitting wafer to be of Pb.

15. The method of claim 11, wherein said radionuclide source is disposed in vivo.

16. The method of claim 15, wherein said radionuclide source is bound to a preferential locator.

17. The method of claim 16, wherein said radionuclide source is bound to said preferential locator, which is one or more of an antibody, an antibody fragment, a single chain antibody, a chimeric antibody, a somatastatin congener, an aptimer, a peptide, or an avimer.

18. The method of claim 11, wherein said radiation-detecting probe instrument is constructed as a finger probe.

19. A method for detecting an external imaging radionuclide source emitting energy above about 80 KeV, wherein said external imaging radionuclide source is bound to a preferential locator that binds to neoplastic tissue, which comprises the steps of:
(a) administering said external imaging radionuclide source bound preferential locator to a patient suspected of having neoplastic tissue;
(b) subjecting said patient to external imaging; and
(c) surgically accessing said patient and using the probe of claim 1 to locate said external imaging radionuclide source.

20. The method of claim 19, wherein said preferential locator is one or more of an antibody, an antibody fragment, a single chain antibody, a chimeric antibody, a somatastatin congener, an aptimer, a peptide, or an avimer.
